(19) United States
(12) Patent Application Publication
(10) Pub. No.: US 2007/0154921 A1
(43) Pub. Date: Jul. 5, 2007

(54) Method and System for Phase-Locked Sequencing

(75) Inventors: Timothy M. Woudenberg, Foster City, CA (US); Meng Taing, Hayward, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/611,339

(22) Filed: Dec. 15, 2006

Related U.S. Application Data
(60) Provisional application No. 60/751,244, filed on Dec. 16, 2005.

(51) Int. Cl. C12Q 1/68; C12M 1/34; G01J 3/42

(52) U.S. Cl. 435/6; 435/287.2; 356/319

(57) ABSTRACT

System and methods according to exemplary embodiments of the present disclosure utilize a sample holder configured to hold at least one confined single-molecule analyte in a solution of labeled nucleotide bases. Each single-molecule analyte has a single template nucleic acid molecule, an oligonucleotide primer, and/or a single nucleic acid polymerizing enzyme. A least one light source is used to illuminate a detection volume around each confined analyte, and a pulsed source sends a pulsed radiation to the at least one detection volume. The timing of incorporation events at the analytes are controlled by the pulsed radiation, and when multiple analytes are provided on the sample holder, the incorporation events at the analytes can be phase locked and synchronized using the pulsed radiation.

### CROSS REFERENCE TO RELATED APPLICATONS

This application claims a priority benefit under 35 U.S.C. § 119(e) from U.S. patent application Ser. No. 60/751,244, filed Dec. 16, 2005, which is incorporated herein by reference.

### FIELD

The present application relates to molecular analysis, and more particularly to single molecule nucleic acid sequencing.

### INTRODUCTION

DNA sequencing allows the determination of the nucleotide sequence of a particular DNA segment. Many conventional DNA sequencing methods use fluorophores to help observe DNA sequencing events. The four nucleotides or dNTPs, which are bases or building blocks of DNA molecules, are labeled with distinguishable fluorescent dyes so that fluorescent signals emitted from different nucleotides can be used to distinguish among them. In a recently envisioned real-time single molecule enzymatic sequencing scheme, attempts are made to observe a single polymerase molecule or enzyme as it adds dNTP bases one at a time to an extending oligonulceotide primer attached to a template single strand DNA molecule. See Levene et al., US Patent Application Publication Number 2003/0174992 A1, which is incorporated herein by reference. Real-time single molecule enzymatic DNA sequencing can be less costly than traditional DNA sequencing techniques, such as the dideoxy sequencing method developed by Fred Sanger, which requires complex sample preparation to produce pure sequences.

In the sequencing technique described by Levene et al, supra, the enzyme-template complex is confined in a detection volume defined by a so-called zero-mode waveguide. The detection volume is small enough (˜1 zeptoliter) so that the fluorescent signals from freely diffusing labeled dNTPs are infrequent and distinct from those emitted from incorporated dNTPs. The occasional visit of a dNTP to the detection volume may be observed as a momentary (˜1 microsecond) burst of fluorescence as it diffuses into and out of the detection volume. Thus, any fluorescence burst of significant duration (e.g., ˜1 millisecond) is deemed to have been originated from a bound dNTP. To prevent dye labels on adjacent incorporated bases from interfering with the observation of a new incorporation event, the dye label on each incorporated dNTP is photo bleached by laser excitation after incorporation is observed. Or, the dNTPs are labeled at the gamma phosphate, which is cleaved by the enzyme during incorporation.

The problem with this approach is that it does not consider the occurrence of false bindings where the enzyme may bind a dNTP for a significant amount of time and then reject it without incorporation. These false bindings may happen more frequently than real incorporations. By simply recording fluorescent bursts of significant durations from an enzyme-template complex, both false bindings and real incorporations are recorded as base incorporation or base calling events and an incorrect sequence is derived. Therefore, for the real-time single molecule enzymatic sequencing scheme to work, one must be able to tell the difference between a false binding and a true incorporation.

### SUMMARY

The present disclosure provides apparatus, systems and method for single molecule nucleic acid sequencing, nucleic acid re-sequencing, and/or detection, and/or characterization of single nucleotide polymorphism (SNP analysis) including gene expression.

A system according to exemplary embodiments of the present disclosure comprises a sample holder having structures formed thereon for defining at least one detection volume each for confining a single-molecule analyte having a single template nucleic acid molecule, an oligonucleotide primer, and/or a single nucleic acid polymerizing enzyme. The system further comprises at least one light source configured to illuminate the sample holder, an optical assembly configured to collect and detect light emissions from the at least one detection volume, and a pulsed source for sending a pulsed radiation, such as pulsed light signals or light pulses, to the at least one detection volume.

The sample holder is configured to hold a solution of labeled nucleotides. In some embodiments, each nucleotide is labeled with a fluorescent dye and has a quencher attached to the gamma phosphate. A true incorporation of the nucleotide results in the release of the gamma phosphate and thus the quencher, causing the fluorescent emission from the fluorescent dye to increase by about 20 fold and providing a clear and unambiguous signal to indicate a base incorporation event.

In other embodiments, each nucleotide is labeled with a bulky label such that when the nucleotide is incorporated into a template nucleic acid molecule, the bulky label substantially slows down subsequent incorporation process at the template molecule. The bulky label is attached to the nucleotide by a photocleavable linker that can be cleaved by one of the light pulses, allowing the bulky label to be removed and the next base to be quickly incorporated after the delivery of the light pulse. Thus, the timing of incorporation events at the analytes can be controlled by the light pulses, and when multiple analytes are provided on the sample holder, the incorporation events at the analytes can be phase locked and synchronized by the light pulses.

These and other features of the present teaching are set forth herein.

### BRIEF DESCRIPTION OF THE DRAWINGS

The skilled artisan will understand that the drawings, described below, are for purposes of illustration only, and are not intended to limit the scope of the present teaching in any way.

FIG. 1 is a layout of a system for single molecule analysis according to exemplary embodiments of the present teaching.

FIG. 2 is a top view of a sample holder in the system.

FIG. 3 is a cross-sectional view of a portion of the sample holder.

FIG. 4A is a 3-dimensional view of a portion of the sample holder according to further embodiments of the present teaching.

FIG. 4B is a diagram illustrating a DNA sequencing process along a channel on the sample holder according to exemplary embodiments of the present teaching.

FIG. 5 is a state diagram illustrating a seven-state mathematical model of a T7 polymerase.

FIGS. 6 and 7 are charts of simulation results using the seven-state mathematical model.

FIGS. 8A and 8B are representative diagrams illustrating the incorporation of a nucleotide labeled with a reporter and having a quencher attached to the gamma phosphate in the nucleotide.

FIGS. 9A and 9B is a diagrams of exemplary chemical structures of labeled nucleotides.

FIGS. 10A to 10C are representative diagrams illustrating the incorporation of a nucleotide with a bulky reporter attached thereto and the cleavage of the bulky reporter with light after the incorporation of the nucleotide.

FIGS. 11A and 11B are diagrams illustrating the incorporation of a nucleotide with an exemplary bulky reporter attached thereto and the cleavage of the bulky reporter with light after the incorporation of the nucleotide.

### DESCRIPTION OF VARIOUS EMBODIMENTS

It is to be understood that both the foregoing summary and the following description of various embodiments are exemplary and explanatory only and are not restrictive of the present teachings. In this application, the use of the singular comprises the plural unless specifically stated otherwise. Also, the use of “or” means “and/or” unless stated otherwise. Similarly, “comprise,” “comprises,” “comprising,” and “including” are not intended to be limiting.

Additionally, while certain embodiments are described in detail herein, particularly embodiments suitable for analysis of single molecule nucleic acid synthesis, it is to be understood the apparatus, systems and methods of the present disclosure may be employed in other applications for analysis of single molecules, such as but not limited to directed resequencing, SNP detection, and gene expression.

Furthermore, the figures in this application are for illustration purposes and many of the figures are not to scale with corresponding hardware or physical entities. Many parts of the features in the figures in this application are drawn out of scale purposefully for ease of illustration.

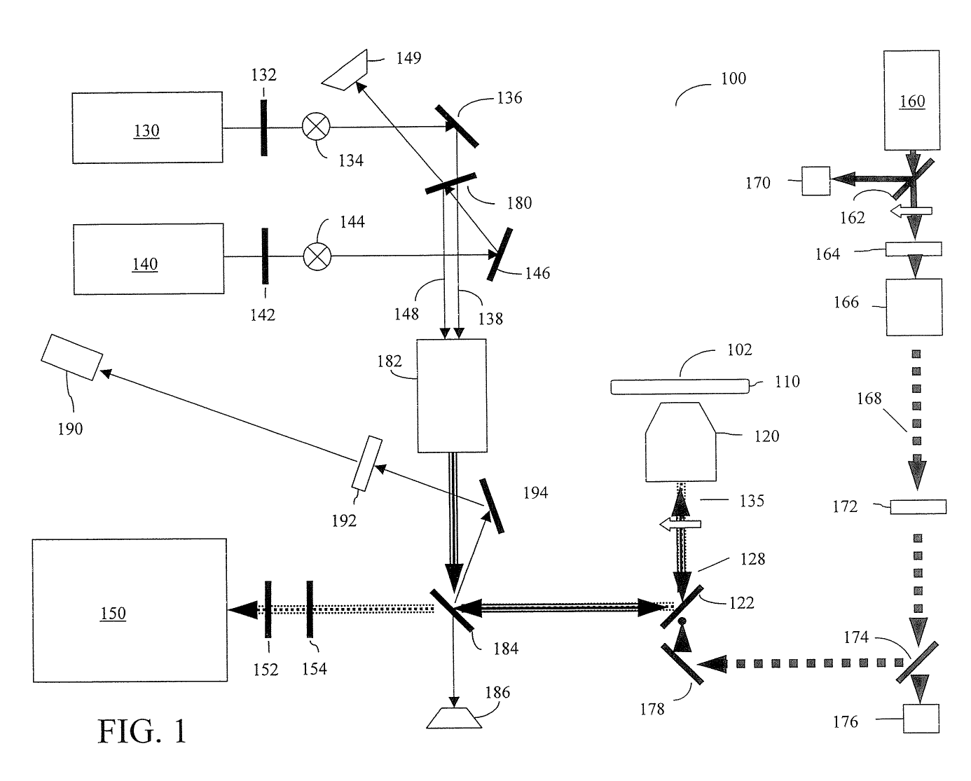

FIG. 1 is a block diagram illustrating a system 100 for single molecule DNA analysis according to an exemplary embodiment of the present teaching. As shown in FIG. 1, system 100 comprises a sample holder 110, an optical objective 120 under the sample holder 110, a first light source 130, an optional second light source 140, a detector 150, and a third light source 160. In one embodiment, when both the first light source 130 and the second light source 140 are provided, they are laser sources of different wavelengths. For example, the first light source 130 may be a 488 nm laser source while the second light source 140 may be a 632.8 nm laser source.

To direct light from light sources 130 and 140 to the sample holder 110, system 100 may further comprise a first optical assembly including, for example, neutral-density (ND) filters 132 and 142 in front of light sources 130 and 140, respectively, polarization filters 134 and 144 in front of ND filters 132 and 142, respectively, broadband (BB) mirrors 136 and 146 in front of polarization filters 134 and 144, respectively, a narrow-passband (NB) filter 180 between BB mirrors 136 and 146, a beam expander 182 in front of the NB filter 180, a wedge mirror 184 in front of the beam expander 182, and an SP mirror 122 between the wedge mirror 184 and the objective 120.

In one embodiment, the laser light from the first light source 130 passes through the ND filter 132, and is polarized by the polarization filter 134 into, for example, circularly polarized light, which is then reflected by the BB mirror 136 towards the NB filter 180. When the second light source 140 is provided, the laser light from the second light source 140 passes through the ND filter 142, and is polarized by the polarization filter 144 into, for example, circularly polarized light, which is reflected by the BB mirror 146 toward the NB filter 180. The NB filter 180 is configured to allow light in a narrow wavelength band around the wavelength of the laser light from the first light source 130 to pass and to reflect light outside the narrow wavelength band. Therefore, the light from the first light source 130 should travel pass through the NB filter 180 and becomes light beam 138 toward the beam expander 182, while most of the light from the second light source 140 is reflected from the NB filter 180 and becomes light beam 148, which joins with the light beam 138 toward the beam expander 182. A beam stop 149 is provided to collect any light from the second light source that has not been reflected by the NB filter 180.

In one embodiment, the beam expander 180 is configured to expand light beams 138 and 148 to about 10 to 20 times their original widths. The expanded light beams 138 and 148 are thereafter reflected by the wedge mirror 184 toward the SP mirror 122, which in turn reflects the light beams toward the sample holder 110 through the objectives 120 as excitation light 135. In one embodiment, the wedge mirror 184 reflects a large percentage, such as 80%, of light beams 138 and 148, while transmitting a small percentage, such as 10%, of the light beams. A beam stop 186 is provided to collect the transmitted portions of the beams 138 and 148.

To detect fluorescent signals from the sample holder 110, system 100 comprises a second optical assembly including, for example, the objective 120, the SP mirror 122, and notch filters 152 and 154. Fluorescent light from the sample holder, together with a portion of the excitation light reflected from surfaces of the sample holder 110, is collected by the objective 120 to form light beams 128, which are reflected by the SP mirror 122 toward the wedge mirror 184. The wedge mirror is configured to allow passage of most of the light beams 128, while reflecting a small portion of the light beams 128 toward a BB mirror 194, which sends the small portion of the light beams 128 toward a focus charge-coupled device (CCD) 190 through lens 192. The small portion of the light beams 128, especially the reflected excitation light contained therein, is used for calibration of the objective 120 and/or the detector 150 to provide better focus of the fluorescent light in the detector 150. The majority of the light beams 128 are directed to the detector 150 through notch filters 152 and 154. Notch filter 152 and 154 are each configured to block a very narrow wavelength range around the wavelength of light beam 138 or 148, respectively, so that the reflected excitation light, or a significant portion of it, does not enter the detector 150.

For purposes discussed below, the third light source 160 is configured to generate laser light or light pulses. As a non-limiting example, the third light source 160 is a 355 tripled YAG laser source configured to generate 355 laser light that is polarized with the electric field direction in the light parallel to the plane of the drawing. System 100 comprises a third optical assembly including, for example, a pellicle beam splitter 162 in front of the third light source 160 that splits the light beam from the third light source into two components, one being directed to a PIN diode 170, and the other being directed to a first set of at least one lens 164. A shutter 166 is provided in front of the lens 164 and is configured to select the light pulses. The selected light pulses 168 pass through a second set of at least one lens 172 toward a mirror 174. The mirror 174 is configured to reflect most of the selected light pulses 168 while allowing a small portion of each pulse to pass and be collected by a PIN diode 176. In one example, the mirror 174 is a 355 nm P-type mirror (mirror 355 nm P) having an associated reflection coefficient dependent on the direction of polarization of the incident light and the reflection coefficient reaches its maximum when the electric field direction in the light pulses 168 is parallel to the plane of incidence, which is the plane formed by the incident light beam and the normal of the mirror and is thus parallel to the plane of the drawing.

The reflected light pulses 168 are further reflected by another mirror 178, which directs the light pulses toward the mirror 122. Mirror 122 while reflecting light in a reflection wavelength range, such as 450-700 nm, which encompasses the wavelength of light sources 130 and 140, is configured to allow passage of the light pulses 168, which in this example has a wavelength of 355 that is outside the reflection wavelength range. The light pulses 168 are therefore directed toward the sample holder 110 through the objective 120.

For ease of illustration, the components of system 100 are drawn on a same plane in FIG. 1. In one exemplary embodiment, most of the optical components including the light sources, 130 and 140, the detector 150, the laser source 160, and the first and third optical assemblies are laid out on a breadboard, while the sample holder 110 is positioned over the breadboard with the objective 120 positioned between the breadboard and the sample holder 110. So, light from the first, second, and third light sources are directed by mirrors 122 and 178 out of the plane of the drawing toward the sample holder. As an example, the objective 120 is a 40× objective, and the mirror 178 is a 355 nm S-type mirror having an associated reflection coefficient dependent on the direction of polarization of the incident light and the reflection coefficient reaches its maximum when the electric field direction in the light pulses 168 is perpendicular to the plane of incidence. Mirror 178 can also be a BB mirror.

PIN diode 170 receives a portion of the light pulses from light source 160 reflected by the pellicle 162 and determines the timing of the light pulses. The timing information is used by shutter 166 to select the light pulses from light source 160 so as to control the time interval between two adjacent pulses in light pulses 168. PIN diode 176 is used to verify that the timing of the shutter 166 is properly controlled.

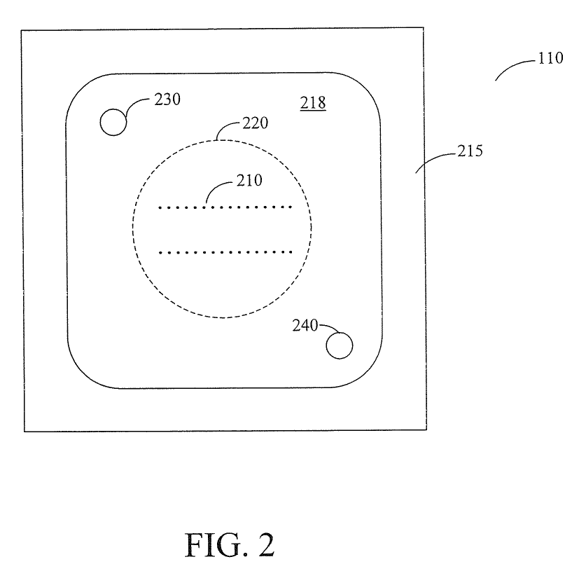

In exemplary embodiments of the present teaching, sample holder 110 has cavities sized less than the wavelengths of light beams 138 and 148 in at least one dimension. FIG. 2 is a block diagram of a top-down view of sample holder 110 according to exemplary embodiments. As shown in FIG. 2, in exemplary embodiments, sample holder 110 is configured to hold at least one spatially constrained single-molecule analyte 210 in a field of view 220 of the objective 120. Sample holder 110 may further comprise a base 215 and a cover 218. A space (not shown) is formed between the cover 218 and the base 215, which space serves as a sample chamber for holding a sample fluid that supplies reactants for the analytes 210. In various embodiments, in applications of nucleic acid sequencing, each single-molecule analyte 210 is an enzyme-template complex having a single polymerase molecule and a single template nucleic acid molecule, or an enzyme-template-primer complex having an oligonulceotide primer attached to the template single strand nucleic acid molecule; and the sample fluid comprises a fluorophore solution of fluorescent-labeled nucleotides. Sample holder 110 may further comprise a fill hole 230 for filling the sample chamber with the sample fluid and a drain hole 240 for draining the sample fluid from the sample chamber. Fill hole 230 and drain hole 240 are preferably located near two opposite corners of sample holder 110, as shown in FIG. 2, for more complete draining and washing away of sample fluid.

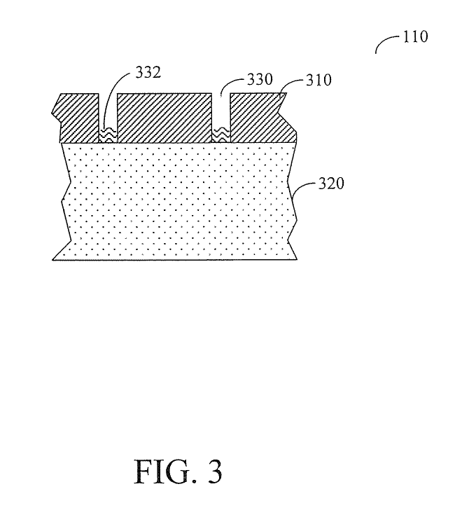

In various embodiments, as shown in FIG. 3, the base 215 of the sample holder includes a film 310 formed on a substrate 320 made of a material transparent to light beams 148 and 138, to light pulses 168, and to the fluorescent emissions from the nucleotides. Film 310 has etched patterns forming cavities or holes 330 for housing the analytes. In some embodiments, these cavities 330 are circular, as shown in a cross-sectional view in FIG. 3, and as described in Patent Application Number U.S. 2003/0174992 by Levene et al. As a specific, non-limiting example, substrate 320 is a fused silica substrate, and film 310 is made of a material opaque to the light beams 148 and 138, such as aluminum or another metallic material. The cavities 330 can be formed by masking and plasma etching the film 310. Each cavity 330 has a diameter that is substantially smaller than the wavelength of either light beam 138 or light beam 148, and a depth that is sufficient to block transmission of the excitation light through the hole. Thus, each cavity 330 acts as a zero-mode waveguide for the excitation light, allowing the excitation light, which comes to the waveguides from the substrate side, to penetrate only a small observation volume 332 near a bottom portion of the cavity 330. At the same time, the zero-mode waveguides also serve to block light emitted or scattered from the sample fluid in the sample holder 110 except emissions coming from any light emitting agents immobilized in the observation volumes 332 in the waveguides or diffusing past the observation volumes 332 in the waveguides.

Thus, in some embodiments, to allow the detection and analysis of light emitted from incorporating nucleotides, the polymerase and/or template nucleic acid molecule in each analyte 210 is immobilized in the observation volume 332 of a zero-mode waveguide 330, so that light emitted from an incorporating nucleotide can escape the cavity 330, pass through the substrate 320 and be collected by the objective 120. Some of the techniques for immobilizing molecules involved in a genetic assay in zero-mode waveguides are described in detail in U.S. Patent Application Number U.S. 2003/0044781 by Korlach et al., which is incorporated herein by reference, and also in commonly owned Provisional Application Attorney Docket Number 347461US/MSS/JJZ (470438-164), which is also incorporated herein by reference.

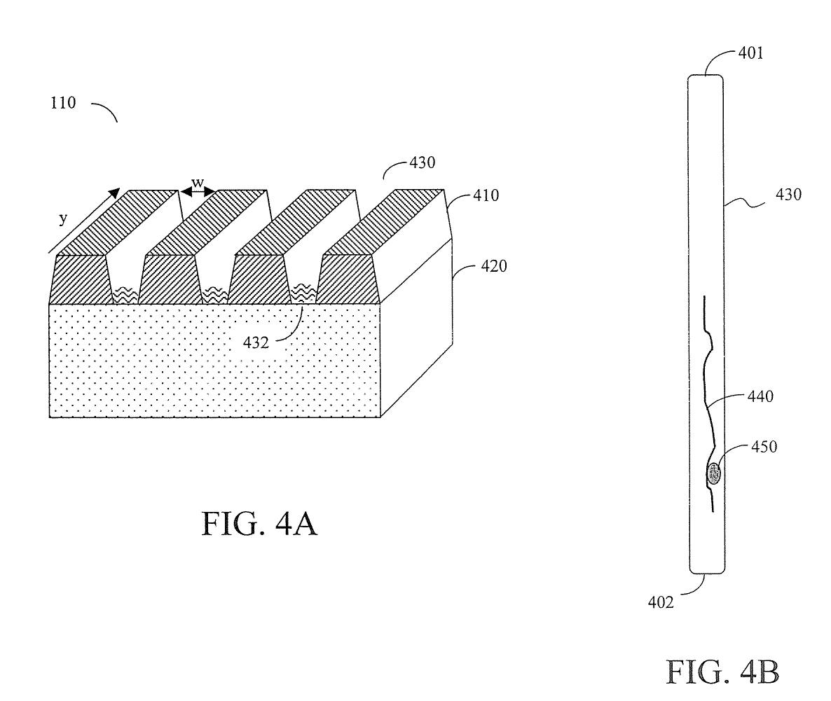

In alternative embodiments of the present teaching, sample holder 110 comprises slots or channels to define at least one observation volume for confining the analytes 210 on the sample holder 110. FIG. 4A illustrates a 3-dimensional view of the sample holder having a film 410 formed on a substrate 420 and a plurality of channels 430 formed in the film 410. As a non-limiting example, film 410 is made of a material opaque to the excitation light, such as aluminum or another metallic material, and substrate 420 is made of a material transparent to the excitation light, such as fused silica. Each channel 430 has a width w that is smaller than the wavelength associated with either light beam 138 or 148. When channels 430 instead of cavities 330 are provided, light beams 138 and 148 are preferably linearly polarized and the polarization direction is oriented such that the electric field vector in the light wave is along the length direction of the channels. Thus, only a small observation volume 432 near a bottom portion in each channel 430 would be illuminated by the excitation light, as shown in FIG. 4A. Channels 610 can be formed using conventional techniques, such as conventional semiconductor processing or integrated circuit (IC) fabrication techniques.

Sample holder 110 with channels 430 formed thereon has multiple advantages over a sample holder with zero-mode waveguide holes 330 formed thereon. Because the fluorescent emissions are largely unpolarized, they would not be attenuated when they try to exit the channels 430 as much as when they try to exit holes 330 of sub-wavelength dimension. So, more emitted light from sample holder 110 can be collected and detected by the objective 120, resulting in increased signal to noise ratio. In addition, as shown in a top-down view of a channel 430 in FIG. 4B, channel 430 can house a larger template molecule 440 if the template molecule 440 is oriented parallel to the channel. This allows the polymerase 450 to migrate down the template 440 for a much longer distance without exiting the illuminated volume 432. The template molecule can be tethered so that it can remain in one location while the polymerase, having a finite processivity, may fall off the template and be replaced by another polymerase. This way, a longer read length can be achieved, which leads to significantly simplified assembly process, especially during denovo sequencing. Although FIG. 4B shows that channel 430 is closed at both ends 401 and 402, each channels 430 on sample holder can be open on either or both ends by extending all the way to the edge(s) of the substrate 420.

The polymerase or template molecules can be attached to sample holder 110 using conventional photoactivatable linkers. In exemplary embodiments of the present teaching, more than one polymerase or template molecules can be attached to sample holder 110 in a resolvable fashion, and each template molecule or an oligonucleotide molecule can be stretching along the bottom surface of a channel 430, as described in commonly-owned Provisional Application Attorney Docket Number 34746/US/MSS/JJZ (470438-164), which has been incorporated herein by reference.

After populating the sample holder 110 with the analytes 210, the sample holder 110 is placed in system 100. A fluorophore solution comprising fluorescence labeled nucleotide analogs is applied to the sample holder 110. The fluorescent label on the nucleotide analog emits fluorescent light upon illumination by the excitation light. In exemplary embodiments of the present teaching, four different nucleotide analogs are labeled with four different fluorescent dyes each having a unique emission spectrum. The four different fluorescent dyes can also be associated with four different frequency bands each corresponding to a peak in emission intensity according to the respective spectrum. The four different frequency bands are hereafter referred to as first, second, third, and fourth frequency bands.

To observe light emitted from each analyte 220, excitation light from light sources 130 and/or 140 is directed towards the substrate side of the sample holder 110, and signals from fluorescing nucleotides are collected by the objective 120 and directed to the detector 150. The fluorescent light signals from multiple analytes 220 on the sample holder 110 can be substantially simultaneously collected and detected, as described in commonly assigned Provisional Application Attorney Docket Number 34746/US/MSS/JJZ (470438-164), which has been incorporated herein by reference. Since only fluorescent signals from the small observation volumes 332 or 430 are observable, as discussed above, and each observation volume is small, fluorescent emissions from freely diffusing labeled dNTPs that make their way to the detector should be infrequent and distinct from those emitted from incorporated dNTPs. Thus, a fluorescence burst of significant duration (e.g., ˜1 millisecond) should be originated from a dNTP bound to an analyte, which is confined in an observation volume. To reduce or eliminate interference between fluorescent signals associated with consecutive incorporation events on a same analyte, after detection of an incorporation event, fluorescent label on the newly incorporated nucleotide can be bleached, cleaved or otherwise removed with a known technique. Photo-cleavable linkers may be utilized to facilitate efficient and consistent removal of the fluorescent labels.

It is found, however, that the duration of a fluorescent burst from a spatially constrained analyte is not sufficient to determine if a nucleotide has been incorporated. There are reasons to believe that more than one mechanism can produce fluorescent bursts of comparable duration to be detected by the detector 150, and these mechanisms must be distinguished in order to yield useful sequencing data. A polymerase enzyme is often visualized as being a machine that chugs through a sequence of steps along a template nucleic acid molecule in an orderly process with roughly fixed timing for every incorporated nucleotide. This visualization, however, is far from being the truth.

To model an incorporation process, a seven-state mathematical model for an enzymatic system involving a T7 polymerase was constructed using an enzyme-modeling computer program, with enzyme rates taken from the literature. See Donlin, Maureen J.; Patel, Smita S.; and Johnson, Kenneth A.; “Kinetic Partitioning between the Exonuclease and Polymerase Sites in DNA Error Correction,” Biochemistry (1991), 30(2), 538-46. See also Wong, Isaac; Patel, Smita S.; and Johnson, Kenneth A.; “An Induced-Fit Kinetic Mechanism for DNA Replication Fidelity: Direct Measurement by Single-Turnover Kinetics,” Biochemistry (1991), 30(2), 526-37.

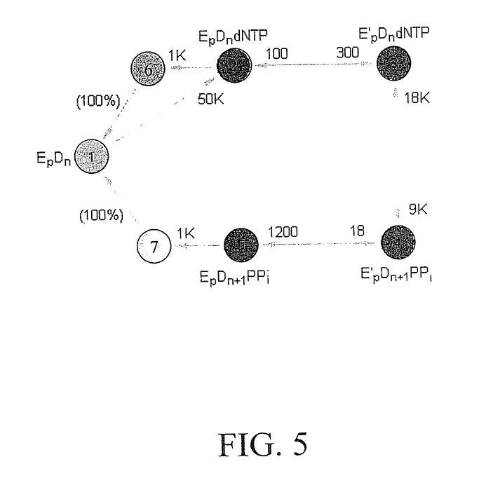

As shown in FIG. 5, the seven states include state 1 representing the enzyme-template-primer complex before and after a modeled incorporation event, states 2-5, which are so called “on” states representing different states of a modeled fluorescent labeled nucleotide bound with the enzyme-template-primer complex, and states 6-7, which are pseudo states inserted in the model for the purposes of tracking exits from the “on” states. Transitions going clockwise in the state diagram are modeled forward reactions toward incorporation and transitions going counter-clockwise are modeled backward reactions toward separation. The transition from state 1 to 2 is a bimolecular reaction. The pseudo first order rate constant associated with the reaction is proportional to the free dNTP concentration in the fluorophore solution, which, in this example, is assumed to be 100 μM. The transition from state 2 to state 3 includes a conformational change in the enzyme and is the rate-determining step for the forward reaction. The transition from state 3 to state 4 is the creation of the covalent bond of the dNTP base and the cleavage of the pyrophosphate. This transition is reversible and does not result in the release of the pyrophosphate. Transition from state 4 to state 5 results in a conformational change of the enzyme. Transition out of State 5 to state 7 results in the release of the pyrophosphate and is irreversible because the pyrophosphate concentration in the ambient is zero or near zero.

While the enzymatic system is constrained such that a transition out of state 2 can only result in state 1 or state 3, the statistics on the trajectory through this process is surprising. After modeling using published kinetic data, it is found that the average duration of the dNTP in the “on” states is about the same whether the dNTP ends up being incorporated into or separated from the enzyme-template complex. This result is contrary to the conventional belief that a productive clockwise exit from the “on” states would, in the large majority of cases, take longer than an unproductive counter-clockwise exit. For example, assume one sets a threshold time of 2.1 msec and regards a dNTP staying in the “on” states longer than this threshold as being incorporated into the template and a dNTP staying in the “on” states shorter than this threshold as being separated from the template, simulation results using the above seven-state model, which are discussed below, suggest that one would get sequencing data that is correct only 55% of the time.

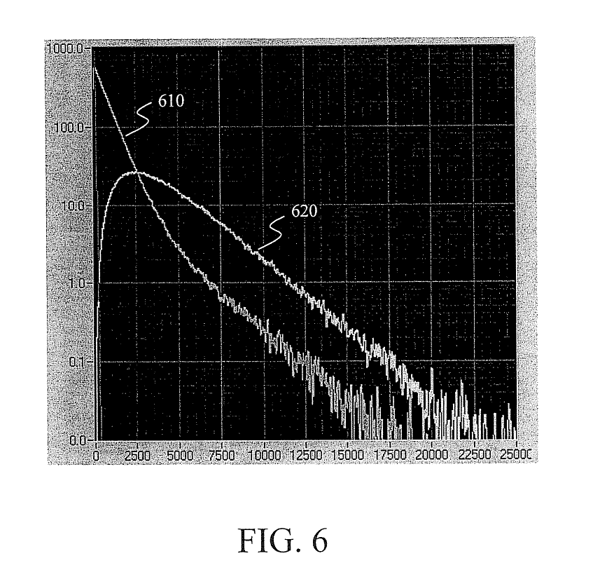

FIG. 6 illustrates the results from a 1000 second simulation run with 1 μsec time slices. The simulation results are represented here as histogram traces 610 and 620 with time spent by the dNTPs in the on states marked in microseconds on the horizontal axis and the logarithmic of the frequency or probability of dNTPs being incorporated into (trace 620) or separated from (trace 610) the enzyme-template complex marked on the vertical (axis). Trace 610 is for unproductive events (dye binding followed by separation), and trace 620 is for productive events (dye binding followed by incorporation).

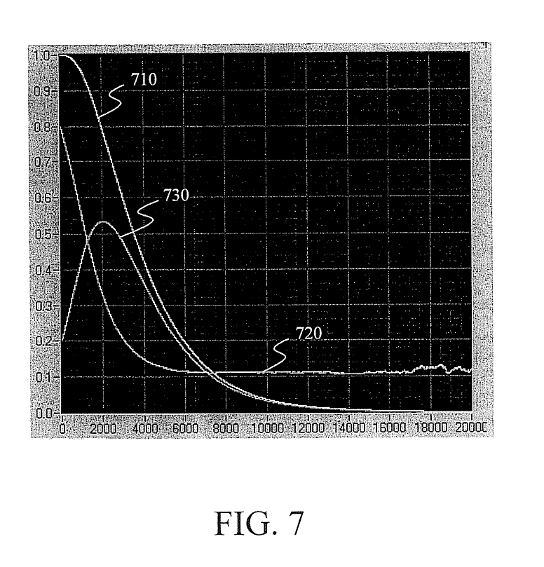

FIG. 7 illustrates base calling accuracy as a function of threshold time in microsecond and includes trace 710 for base calling efficiency, trace 720 for error rate, and trace 730 for accuracy rate. For example, for a selected threshold time T, the base calling efficiency BE is defined as the probability that a true incorporations would take at least that long to occur, and can be expressed mathematically as:

$$B E ( T ) = {\sum }_{t = T}^{t = T max} P E ( t ) / {\sum }_{t = 0}^{t = T max} P E ( t )$$

where PE(t) represents the probability of a dNTP being incorporated after spending a period of time t in the “on” states (trace 620), and Tmax is the predetermined maximum time, which in one example is set to be 25000 μsec. In other words, BE(T) is equal to a first normalized area under the trace 620 from a time equal to the threshold time T to the predetermined maximum time Tmax. Thus, for a threshold time of 0 μsec, the base calling efficiency is 1 because all of the incorporated dNTPs would have spent longer than 0 μsec in the “on” states.

Likewise, the error rate ER(T) for trace 720 is the rate of error by regarding all dNTPs spending least time T in the “on” state as incorporated, and in one example is computed as a second normalized area under the trace 610 from a time equal to the threshold time to the predetermined maximum time divided by the sum of the first and second normalized areas. Expressed mathematically,

$$E R ( T ) = \frac{{\sum }_{t = T}^{t = T max} U E ( t ) / {\sum }_{t = 0}^{t = T max} U E ( t )}{{\sum }_{t = T}^{t = T max} U E ( t ) / {\sum }_{t = 0}^{t = T max} U E ( t ) + {\sum }_{t = T}^{t = T max} P E ( t ) / {\sum }_{t = 0}^{t = T max} P E ( t )}$$

where UE(t) represents the probability of a dNTP being separated from the target after spending a period of time t in the “on” states (trace 610).

The accuracy rate AR(T) for trace 730 represents the accuracy of sequencing data obtained by considering all dNTPs spending at least time T in the “on” states as incorporated dNTPs. AR(T) should depend on both the error rate ER(T) and the base-calling accuracy BE(T). In one example, the accuracy rate ER(T), as plotted with trace 730 in FIG. 7, is computed as:
AR(T)=BE(t)[1−ER(T)],
As shown by trace 730 in FIG. 7, the best accuracy of sequencing data obtained by using threshold time to determine whether a dNTP is incorporated occurs when the threshold time is set to be 2.1 millisecond, but this best accuracy is less than about 55%.

What is needed is a signal that unambiguously indicates incorporation. There is some effort in this area by Susan Harding of Visigen to create a FRET labeled polymerase enzyme that allows the conformation of the enzyme be directly monitored. Nonetheless, this scheme, even if it worked perfectly, would still not bring certainty to the issue. There is also some well-founded doubt that this scheme could be made to provide good enough signal-to-noise ratio of the enzyme configuration.

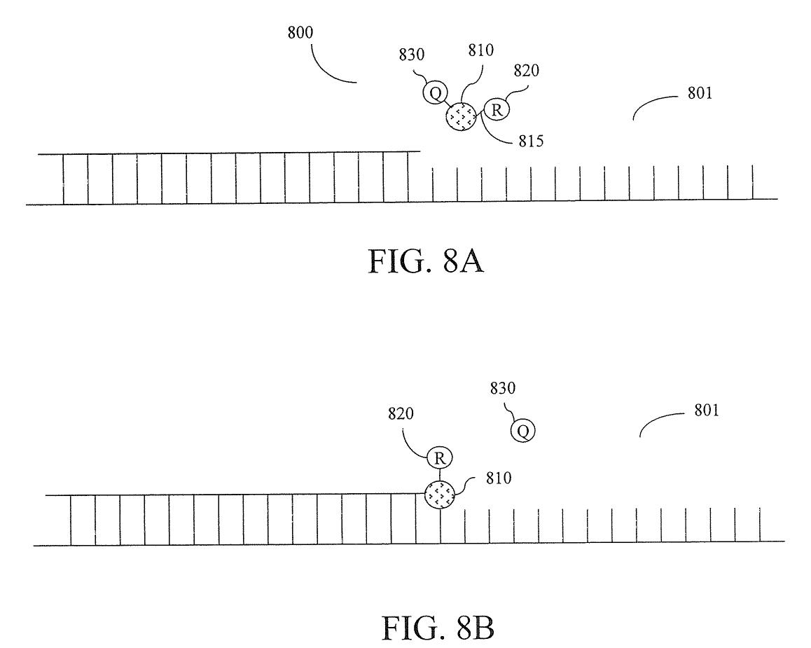

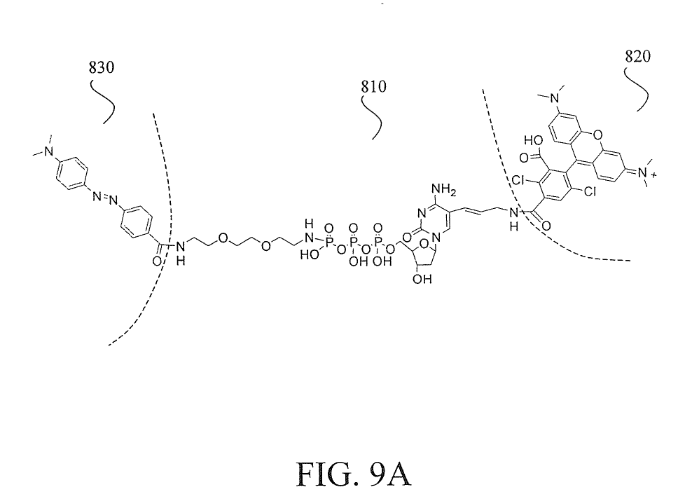

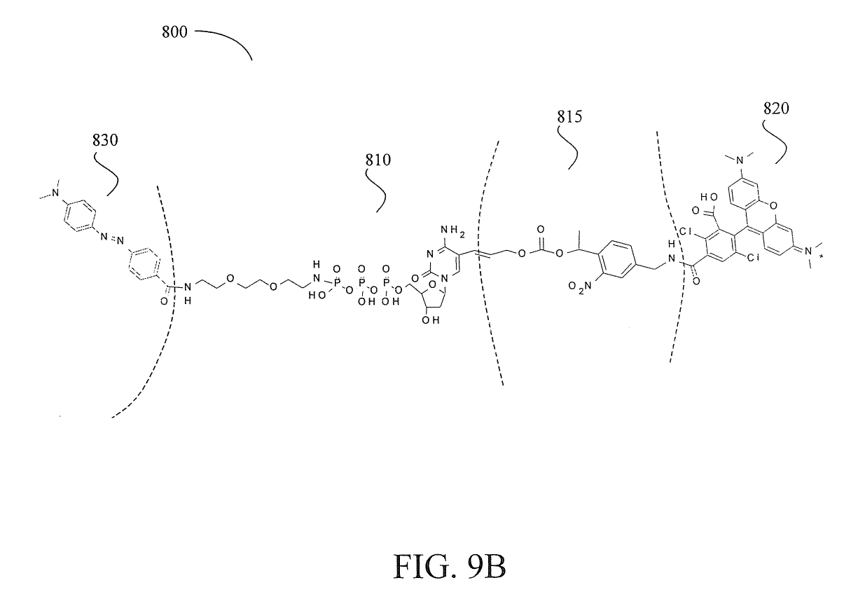

To solve the ambiguity problem discussed above, in one embodiment of the present teaching, as illustrated in FIGS. 8A and 8B, the nucleotides or dNTPs 810 in the fluorophore solution is doubly labeled with a fluorescent reporter 820 and a quencher 830. The quencher 830 is attached to the gamma phosphate of the dNTP 810 such that it is released upon incorporation of the dNTP 810 into an enzyme-template -primer complex 801. Because there is almost zero free pyrophosphate concentration in the ambient, this process is irreversible. The fluorescent reporter is attached to the nucleotide and remains so after incorporation. Thus, an approximately 20 fold increase in fluorescence can be seen from the dNTP 810 when an irreversible process of incorporation liberates the quencher 830, providing a clear and unambiguous signal that a base has been incorporated. An example of a doubly labeled dNTP is shown in FIG. 9A. After detection of the incorporation process, the reporter 820 should be removed or photo-bleached before the next incorporation event so that the detection of the subsequent addition of a base is not influenced by the close proximity of the current reporter.

In some embodiments, the fluorescent reporter 820 is attached to the dNTP 810 via a photocleavable linker (PCL) 815, as shown in FIG. 8A. An example of a PCL dye-quencher dNTP is shown in FIG. 9B. Photocleavable linker 815, such as the one shown in FIG. 9B, allows easy removal of the reporter 820 by light after the incorporation process.

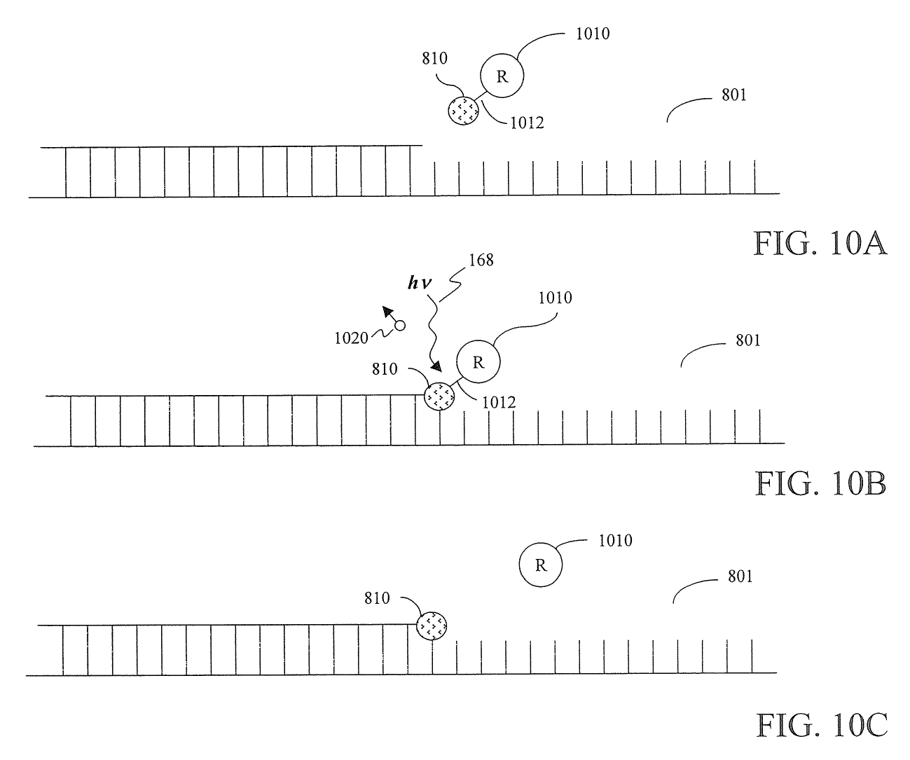

In further embodiments of the present teaching, as illustrated in FIGS. 1 and 10A-10C, external signals, such as the light pulses 168, are used to phase-lock the incorporation cycles with one pulse per base incorporation. As shown in FIG. 10A, the dNTPs 810 are modified such that each has a relatively bulky reporter 1010 attached thereto through a linker 1012 that is cleavable by an external signal, such as one of the light pulses 168. As the polymerase extends a primer template complex 801 by incorporating the dNTPs 810, the reporter on each newly incorporated dNTP 810 acts as an obstacle or impeder to block subsequent incorporation (FIG. 10B). The reporter can be removed to enable the next incorporation when an external signal, such as a light pulse 168, hits the photocleavable link 1012 (FIG. 10C). Once the label is cleaved, it rapidly diffuses away from the enzyme-template-primer complex, out of the detection volume 332 or 432. The enzyme-template-primer complex is then able to rapidly incorporate the next base, as the impeding label is now absent.

The timing of the pulses is important. Each pulse should arrive after the signal from a labeled base 810 has been around long enough, such as more than 20 or 25 millisecond, to indicate that incorporation should have occurred. As an example, the light pulses 168 are used as the external signals and the shutter 166 can be adjusted to control the time separation Δt between adjacent pulses. Thus, with the use of a relatively bulky label that remains attached to the base until the signal is given, the timing of the single molecule enzymatic process can be controlled such that there is either one or zero bases added per each light pulse 168 with little ambiguity over the result.

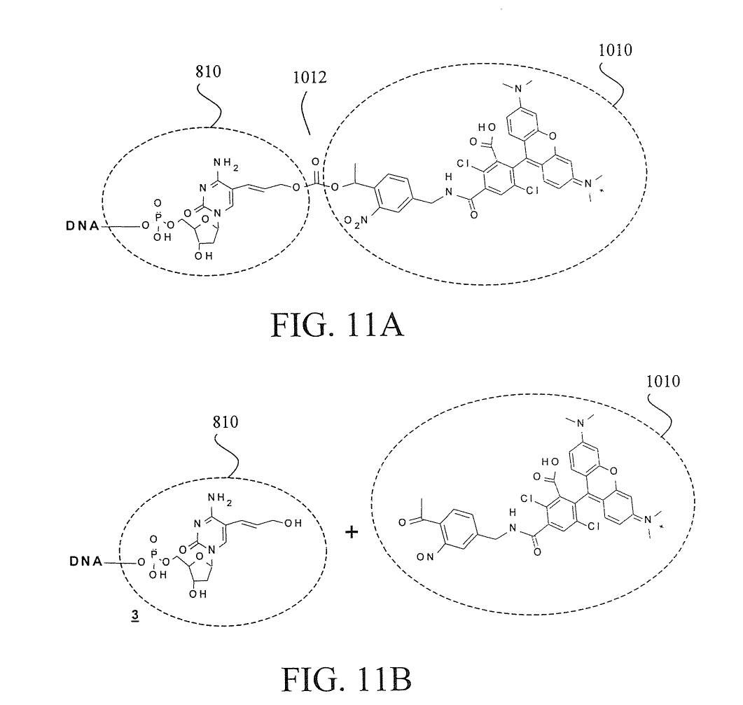

As discussed above, the label 1010 serves two purposes: 1) it signifies that the dNTP is bound to the enzyme-template-primer complex; 2) it significantly impedes the incorporation of the next base. Many types of conventional labels and linkers can be used as the label 1010 and liner 1012. For optimal result, the label 1010 and linker 1012 should be selected such that upon cleavage of the linker 1012, the dNTP would allow quick incorporation of the next base. As an example, the reporter 820 and part or all of the photocleavable linker 815 in the PCL dye-quencher dNTP 800 shown in FIG. 9B can serve together as the bulky reporter 1010 and linker 1012. After the dNTP 810 is incorporated by polymerase into an extending DNA strand 1020, the link 1012 can be cleaved by UV irradiation, as shown in FIGS. 11A and 11B. In this example, upon removal of the bulky reporter 1010 by light emission, a smaller hydroxyallyl substituent that is a neutral non-charged functional group is imparted. This would allow speedy incorporation of another dNTP by polymerase. If the bulky reporter 1010 is not removed, incorporation of another dNTP will be hindered.

The phase-locking technique discussed above is different from prior art stepwise enzymatic sequencing, of which there are many examples. See H. Ruparel et al., “Design and Synthesis of a 3′-O-allyl Photocleavable Fluorescent Nucleotide as a Reversible Terminator for DNA Sequencing by Synthesis,” PNAS, Apr. 26, 2005, vol. 102, no. 17, 5932-5937. The basic limitation of the prior art enzymatic sequencing is that it must be stopped at each base addition so that the last base can be observed. In most cases, this is done with a reversible inhibitor that modifies or protects the 3′ hydroxyl so that a subsequent base cannot be added until the previously incorporated base is observed and the inhibitor removed. This is a good idea in theory but performs badly in practice because either the inhibition or the removal of the inhibition is not 100% effective. In the single molecule enzymatic sequencing case, a failure of inhibition would result in a read error, and a failure to remove the inhibition would result in an end of read. In an ensemble case, a failure of inhibition or removal of inhibition would contribute to dephasing of the population within one sample. In general, the less than perfect efficiency of the inhibitor in the prior art enzymatic sequencing has an overall effect of short read lengths, typically 5 to 25 bases, and poor reliability of results.

In contrast, in the embodiments of the present teaching, no inhibitor is used. Instead, an impeder 1010 is used which does not prevent the addition of a subsequent base, but merely slows it down until the impeder 1010 is removed. In the case that the impeder 1010 is not removed, the addition of the subsequent base happens anyway in a slower pace.

Real time single molecule enzymatic DNA sequencing has the potential of higher speed, higher throughput, and longer read length than traditional DNA sequencing techniques. For higher throughput, a plurality of analytes 210 can be observed substantially simultaneously, as discussed in the commonly owned Provisional Application Attorney Docket Number 34746/US/MSS/JJZ (470438-164), which has been incorporated herein by reference. To image a large number of analytes 210 in a sub-millisecond time frame may pose a challenge to many detection systems, especially when incorporation events in the plurality of analytes occur asynchronously. For example, when charge coupled devices (CCD) are used in the detector 150, a frame rate of above 1 KHz is often required but is difficult to achieve. By labeling the nucleotides with the inhibitors 1010 and using the light pulses 168 to control the timing of the incorporation events in the plurality of analytes, the fluorescent bursts that indicate incorporation from the plurality of analytes are synchronized to the light pulses 168. As a result, a less complicated opto-mechanical system is required to observe the incorporation events from a large number of single-molecule analytes.

In summary, the present teaching includes an apparatus for sequencing a target nucleic acid molecule. The apparatus comprises a sample holder configured to hold a solution including fluorescence-labeled nucleotide bases and to separate and confine at least one single-molecule analyte each comprising a single target nucleic acid molecule and a single nucleic acid polymerizing enzyme. The apparatus further comprises at least one first light source configured to produce excitation light directed toward the sample holder. The excitation light illuminates a small volume around each confined analyte. The apparatus further comprises a second light source configured to produce light pulses for controlling the timing of incorporation events occurring at the at least one analyte.

In exemplary embodiments, the second light source includes a shutter configured to control time separation of adjacent light pulses. The time separation is controlled such that a light pulse is directed to the at least one analyte after a newly incorporated nucleotide at the at least one analyte has been fluorescing for longer than a predetermined time period. The predetermined time period may be about 20-25 milliseconds.

In exemplary embodiments, the nucleotide bases are each labeled with a bulky label such that when the nucleotide is incorporated into the target nucleic acid molecule, a subsequent incorporation event is slowed down by the presence of the bulky label until the bulky label is removed. The bulky label may include a photocleavable linker and a fluorescent dye.

In further embodiments, the timing of incorporation events are controlled such that either one or zero nucleotide base is incorporated at each analyte per each light pulse. The sample holder is configured to confine and separate a plurality of single-molecule analytes, and the light pulses synchronize incorporation events at the plurality of analytes. Each analyte includes a labeled nucleotide, which comprises a nucleotide; a fluorescent label; and a photocleavable linker between the nucleotide and the fluorescent label. The photocleavable linker is selected to allow cleavage of the linker and the label by light after the nucleotide is incorporated, and to allow the next incorporation event to happen after the cleavage. The labeled nucleotide may further include a quencher attached to the gamma phosphate of the nucleotide.

The present teaching further includes a method for sequencing a target nucleic acid molecule, comprising the steps of: providing at least one confined single-molecule analyte in a solution including fluorescent labeled nucleotide bases, each single-molecule analyte comprising a single one of the target nucleic acid molecule and a single one of a nucleic acid polymerizing enzyme; directing excitation light from at least one light source toward the at least one analyte, the excitation light illuminating a small volume around each analyte; and projecting a train of light pulses toward the at least one analyte to control the timing of incorporation events occurring at the at least one analyte.

In exemplary embodiments, the step of projecting includes using a shutter to control time separation of adjacent light pulses. In further embodiments, the time separation is controlled such that a light pulse is directed to the at least one analyte after a newly incorporated nucleotide at the at least one analyte has been fluorescing for longer than a predetermined time period. The predetermined time period may be about 20-25 milliseconds.

In exemplary embodiments, the light pulses are ultraviolet, and the excitation light is circularly polarized.

In exemplary embodiments, the providing step includes labeling the nucleotide bases with bulky labels such that when a nucleotide is incorporated into the target nucleic acid molecule, a subsequent incorporation event is slowed down by the presence of its bulky label. In further embodiments, the bulky label is coupled with the nucleotide by a photocleavable linker such that the bulky label can be cleaved by one of the light pulses.

In exemplary embodiments, the step of projecting includes controlling the timing of the light pulses such that one nucleotide base is incorporated at each analyte per each light pulse, and the step of providing includes providing a plurality of confined and separated single-molecule analytes so that the light pulses synchronize incorporation events at the plurality of analytes.

The foregoing descriptions of specific embodiments of the present teaching have been presented for purposes of illustration and description. They are not intended to be exhaustive or to limit the teaching to the precise forms disclosed, and obviously many modifications and variations are possible in light of the above teaching. The embodiments were chosen and described in order to best explain the principles of the teaching and its practical application, to thereby enable others skilled in the art to best use the teaching and various embodiments with various modifications as are suited to the particular use contemplated. It is intended that the scope of the teaching be defined by the claims appended hereto and their equivalents.

1. An apparatus for sequencing a target nucleic acid molecule, comprising: a sample holder configured to hold a solution including fluorescence-labeled nucleotide bases, and to separate and confine at least one single-molecule analyte each comprising a single target nucleic acid molecule and a single nucleic acid polymerizing enzyme; at least one first light source configured to produce excitation light directed toward the sample holder, the excitation light illuminating a small volume around each confined analyte; and a second light source configured to produce light pulses for controlling the timing of incorporation events occurring at the at least one analyte.

2. A method for sequencing a target nucleic acid molecule, comprising: providing at least one confined single-molecule analyte in a solution including fluorescent labeled nucleotide bases, each single-molecule analyte comprising a single one of the target nucleic acid molecule and a single one of a nucleic acid polymerizing enzyme; directing excitation light from at least one light source toward the at least one analyte, the excitation light illuminating a small volume around each analyte; and projecting a train of light pulses toward the at least one analyte to control the timing of incorporation events occurring at the at least one analyte.
